(19) United States
(12) Patent Application Publication
(10) Pub. No.: US 2005/0181443 A1
(43) Pub. Date: Aug. 18, 2005

(54) Coreactant-including electrochemiluminescent compounds, methods, systems and kits utilizing same

(76) Inventors: Ji Sun, Potomac, MD (US); Pam Liang, Baltimore, MD (US); Mark T. Martin, N. Bethesda, MD (US); Liwen Dong, Rockville, MD (US)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/108,840

(22) Filed: Apr. 19, 2005

Related U.S. Application Data
(63) Continuation of application No. 09/742,033, filed on Dec. 20, 2000.
Application No. 09/742,033 is a continuation of application No. 08/936,971, filed on Sep. 25, 1997, now abandoned.
Application No. 08/936,971 is a continuation-in-part of application No. 08/467,712, filed on Jun. 6, 1995, now Pat. No. 6,852,502.
(63) Continuation-in-part of application No. 08/928,075, filed on Sep. 11, 1997, now Pat. No. 6,524,865.
Application No. 08/928,075 is a continuation of application No. 08/484,766, filed on Jun. 7, 1995, now abandoned.
(63) Continuation-in-part of application No. 08/880,209, filed on Jun. 23, 1997, now Pat. No. 6,165,708.
Application No. 08/880,209 is a continuation of application No. 08/485,419, filed on Jun. 7, 1995, now Pat. No. 5,643,713.
(63) Continuation-in-part of application No. 08/880,210, filed on Jun. 23, 1997, now Pat. No. 6,120,986.
Application No. 08/880,210 is a continuation of application No. 08/368,429, filed on Jan. 4, 1995, now Pat. No. 5,641,623.
(63) Continuation-in-part of application No. 08/880,353, filed on Jun. 23, 1997, now Pat. No. 6,316,180.
Application No. 08/880,353 is a continuation-in-part of application No. 08/485,419, filed on Jun. 7, 1995, now Pat. No. 5,643,713.

(51) Int. Cl. C12Q 1/68; G01N 33/53; C07H 21/04; C12M 1/34

(52) U.S. Cl. 435/6; 435/7.1; 435/287.2; 530/400; 536/25.32

(57) ABSTRACT

A method of generating a electrochemiluminescent emission, which comprises exposing an electrochemiluminescent label linked to a coreactant, to conditions suitable for inducing electrochemiluminescence; said compound; a system for generating an electrochemiluminescent emission, which comprises said compound, means for exposing said compound to electrochemical energy, and means for detecting or measuring luminescence emitted from said compound or a composition containing same; and a kit for performing an assay using said compound.

This application is a continuation-in-part of U.S. application Ser. No. 08/484,766, filed Jun. 7, 1995, U.S. application Ser. No. 08/880,209, filed Jun. 23, 1997, U.S. application Ser. No. 08/880,210, filed Jun. 23, 1997, and U.S. application Ser. No. 08/880,353, filed Jun. 23, 1997.

### FIELD OF THE INVENTION

The present invention is directed generally to analytical chemistry including biochemistry, and to method, compound, system and kit embodiments for effectively generating electrochemiluminescence in connection therewith.

### BACKGROUND OF THE INVENTION

An ever-expanding field of applications exists for rapid, highly specific, sensitive, and accurate methods of detecting and quantifying chemical, biochemical, and biological substances. Because the amount of a particular analyte of interest in a typical biological sample is often quite small, the improvement of assay performance characteristics such as sensitivity is important.

One approach to improving assay sensitivity is to make use of the techniques available for the highly sensitive detection of light (e.g., photomultiplier tubes, photodiodes, avalanched photodiodes, CCD cameras, etc.). In this regard, the use of luminescent indicator molecules is of interest. For example, luminescent labels associated with an analyte of interest (or the binding partner of an analyte of interest) can be used to detect quantitatively the presence of said analyte (or binding partner). Similarly, the amount of an analyte can be determined quantitatively when said analyte participates in a reaction that leads to the modulation of luminescence as illustrated by the following examples: i) the analyte may react with another species thus modulating the luminescent properties of said second species; ii) the analyte may undergo a chemical transformation that modulates the luminescent properties of the analyte itself; iii) the analyte may be a catalyst (e.g., an enzyme) that catalyzes a reaction that leads to the reaction of another species, thus modulating the luminescent properties of said second species and/or iv) the analyte may participate in a reaction that produces a species that then participates in subsequent reactions that lead to a modulation of luminescence. Techniques that have been used to detect luminescent indicator molecules include photoluminescence, chemiluminescence, and electrochemiluminescence (ECL).

Luminescence occurs when a molecule in an electronically excited state relaxes to a lower energy state by the emission of a photon. In photoluminescence (e.g., fluorescence and phosphorescence) an electronically excited state is generated by the illumination of a molecule with an external light source. In chemiluminescence, the excited state is generated as a result of a chemical reaction. In electrochemiluminescence, the electronically excited state is generated upon exposure of the molecule (or a precursor molecule) to electrochemical energy in an appropriate surrounding chemical environment.

The signal in each of such luminescent techniques can be very effectively detected through the use of known instruments. However, the manner in which the luminescent species is generated differs greatly from one to another of those processes. Such differences account for the substantial advantages as a bioanalytical tool that electrochemiluminescence (hereinafter, sometimes “ECL”) enjoys vis-a-vis photoluminescence and chemiluminescence, including: (1) simpler, less expensive instrumentation; (2) stable, nonhazardous labels; and (3) increased assay performance characteristics such as lower detection limits, higher signal to noise ratios, and lower background levels.

Certain applications of ECL have been developed and reported in the literature. U.S. Pat. Nos. 5,147,806; 5,068,808; 5,061,445; 5,296,191; 5,247,243; 5,221,605; 5,238,808, and 5,310,687, the disclosures of which are incorporated by reference, detail various inventions in the field of ECL, and associated advantages. Moreover, U.S. Pat. No. 5,641,623, the disclosure of which is likewise incorporated by reference, details certain aspects of ECL in connection with beta-lactam and beta-lactamase, neither of which is linked to an electrochemiluminescent compound.

The analytical applications of ECL have been reviewed by Knight et al., 1994, Analyst, 119:879-890; Greenway, 1990, Trends in Analytical Chemistry 9:200-203; and Yang et al., 1994, Bio/Technology 12:193-194; these references are similarly incorporated by reference.

But, while the aforementioned ECL techniques are advantageous, further improvements in the effectiveness of ECL embodiments involving more conventional coreactant species would be desirable.

### OBJECTS OF THE INVENTION

One object of the invention is to provide methods for generating electrochemiluminescence utilizing an electrochemiluminescent label, said label being linked to a suitable coreactant, or to a precursor of said coreactant.

Also, it is an object of the invention is to provide ECL methods having improved assay performance characteristics.

Another object of the invention is to provide a compound comprising an electrochemiluminescent label, said label being linked to a suitable coreactant or to a precursor of said coreactant. Additionally, it is an object to provide a kit utilizing said compound and adapted for use in performing ECL assays.

Yet another object of the invention is to provide ECL compounds, and kits utilizing same, having improved assay performance characteristics.

A further object of the invention is to provide systems comprising an electrochemiluminescent label, said label being linked to a suitable coreactant, or to a precursor of said coreactant, and equipment for inducing electrochemiluminescence which can be detected or measured.

A still further object of the invention is to provide ECL systems with improved assay performance characteristics.

Still another object of the invention is to provide methods for generating electrochemiluminescence utilizing an electrochemiluminescent label containing a coordinate complex of a metal, said label being linked to a species which upon exposure to electrochemical energy forms an electrochemiluminescence coreactant, or to a precursor of said species.

And, another object of the invention is to provide a compound comprising an electrochemiluminescent label containing a coordinate complex of a metal, said label being linked to a species which upon exposure to electrochemical energy forms an electrochemiluminescence coreactant or to a precursor of said species. Additionally, it is an object to provide a kit utilizing said compound and adapted for use in performing ECL assays.

Yet a further object of the invention is to provide systems comprising an electrochemiluminescent label containing a coordinate complex of a metal, said label being linked to a species which upon exposure to electrochemical energy forms an electrochemiluminescence coreactant, or to a precursor of said species, and equipment for inducing electrochemiluminescence which can be detected or measured.

### SUMMARY OF THE INVENTION

As used herein, the term “electrochemiluminescent label” (hereinafter sometimes “EL”) encompasses luminescent molecules capable of generating ECL upon exposure to suitable conditions, as well as precursors to said molecules. Such conditions are, for instance, the presence of a coreactant species and the introduction of an amount of electrochemical energy effective to oxidize or reduce the label such that it can interact with the coreactant so as to place the label in an electronically excited state capable of luminescing.

Additionally, as used herein, the term “coreactant” (hereinafter sometimes “CR”) encompasses species which themselves are capable of interaction with an electrochemiluminescent label to result in electrochemiluminescence, precursor species which upon exposure to electrochemical energy are transformed into such interactive species, and species which are capable of undergoing a chemical transformation to form said interactive species or said precursor species.

In one aspect the present invention is directed to a method of generating an electrochemiluminescent emission which comprises exposing an electrochemiluminescent label, said label being linked to a suitable coreactant, to conditions suitable for inducing electrochemiluminescence.

In a further aspect the invention is directed to a compound which comprises an electrochemiluminescent label, which label is linked to a suitable coreactant, such that said compound electrochemilumineses when exposed to electrochemical energy.

In still another aspect the invention is directed to a system for generating an electrochemiluminescent emission, which comprises
(a) a compound which comprises an electrochemiluminescent label, which label is linked to an electrochemiluminescence coreactant;
(b) means for exposing said compound to electrochemical energy; and
(c) means for detecting or measuring luminescence emitted from said compound or a composition containing same.

In yet another aspect the invention is directed to a kit for use in performing an electrochemiluminescent assay for an analyte of interest, said kit having a plurality of solutions each containing a different amount of a compound which comprises an electrochemiluminescent label, which label is linked to a suitable coreactant.

The invention confers significant advantages on its practitioner, such as efficiency and convenience. Further, the invention meets the art's desire for improved assay performance characteristics of the measured species such as signal output, detection limits, sensitivity, etc. This is because the EL and the CR are linked to one another and thus are maintained in effective proximity.

### BRIEF DESCRIPTION OF THE DRAWINGS

FIG. 1 depicts the reaction scheme for synthesizing a conjugate of an electrochemiluminescent label and tripropylamine in accordance with the invention.

FIG. 2 depicts the structures of various tertiary amines utilized in the Examples hereinafter.

FIG. 3 depicts, in chart form, electrochemiluminescent assay results generated utilizing free label and various unconjugated coreactants.

FIG. 4 depicts, in chart form, electrochemiluminescent assay results generated utilizing various label-coreactant conjugates.

FIG. 5 depicts, in chart form, comparative electrochemiluminescent assay results generated utilizing on the one hand free label and unconjugated TPA coreactant, and on the other hand a label/TPA coreactant conjugate.

### DESCRIPTION OF CERTAIN PREFERRED EMBODIMENTS

A central feature of the present invention is the provision and utilization of detectable compounds comprising (A) a suitable coreactant, (B) linked to (C) an electrochemiluminescent label. A further central feature of the invention is that the CR remain covalently linked to the EL throughout all of the postulated reactions. Such compounds and their uses in accordance with the invention afford significant advantages because the EL and the CR are linked to, and thus maintained in effective proximity of, one another such that their interaction to produce electrochemiluminescence is greatly facilitated. For example, because of such components' linkage in proximity (hereinafter sometimes referred to as “effective interactive proximity”) they are reliably and efficiently presented to one another for interaction at the desired time, and the efficacy of the interaction is increased. The salient features of the components of the inventive compounds are described hereinafter along with certain uses of the compounds.

(A) The coreactant

The CR advantageously is a reductant or oxidant capable of intramolecularly providing an electron to or accepting an electron from the EL, so the EL is converted into an excited (i.e., emissive) state.

In certain good embodiments of the invention, the EL is linked to an electrochemiluminescence reactant (hereinafter sometimes “ECR”), which category encompasses (and which term shall herein mean) species which themselves are capable of interaction with an EL to result in electrochemiluminescence, and precursor species which upon exposure to electrochemical energy are transformed into such interactive species. The ECR moiety is suitably either a reductant or reductant precursor as discussed above, or an oxidant or oxidant precursor. The selection depends on the nature of the EL and whether it is to be oxidized or reduced by the conditions selected for the ECL event.

Thus, if the EL is to be oxidized by exposure to electrochemical energy, then the ECR is a reductant or reductant precursor. On the other hand, if the EL is to be reduced, then the ECR is an oxidant or oxidant precursor.

Some examples of known CR species include amines, peroxides, persulfates, oxalates and cofactors (e.g., NADH). Other examples are known in the art; for example, see the previously described references and patents incorporated by reference. Preferably the coreactant has a functional group that allows it to be chemically attached to the EL through a linker. Alternatively, the coreactant may be an integral part of the EL or the linker group. Alternatively, a derivative of a known coreactant is prepared that includes a functional group for linking said coreactant to a linking group or EL. The identify of such functional groups (e.g., amines, carboxylic acids, hydroxides, thiols) will be obvious to those of ordinary skill in the art.

Coreactants are often oxidant or reductant precursors. Typically, said precursors undergo oxidation or reduction at an electrode during the ECL process so as to form strong oxidants or reductants. For example, an ECR may undergo a one-electron oxidation at an electrode to form a strong reductant that then is further oxidized by reaction with an EL to generate ECR.

In certain preferred embodiments of the present invention an amine or amine moiety (of a larger molecule) is utilized as an ECR which can be oxidized to convert it to a highly reducing species. While not wishing to be bound by a theoretical explanation of reaction mechanism, it is postulated that the amine or amine moiety is oxidized by electrochemical energy introduced into the reaction system. The amine or amine moiety loses one electron, and then deprotonates, or rearranges itself, into a strong reducing agent. This agent interacts with the oxidized ECL label comprising a coordinate complex of a metal and causes it to assume an excited state, in which condition it luminesces. The amine or amine moiety preferably has a carbon-centered radical with an electron which can be donated from such carbon, and an alpha carbon or conjugated carbon which can then act as a proton donor during deprotonation in order to form the reductant. The reductant provides the necessary stimulus for converting the ECL label to its excited state, from which electromagnetic radiation is emitted.

The reductant formed from the amine or amine moiety typically has a redox potential, Eₐ, which is defined in accordance with the following formula:
Eₐ≦−hc/λ+K+Eₘ.
In the formula, h is Planck's constant, c is the speed of light, A is the wavelength characteristic of radiation emitted from the excited state of the metal-containing luminophore, K is the product of (i) the absolute temperature (in degrees Kelvin) of the environment in which the ECL interaction takes place and (ii) the change in entropy as a result of the ECL reaction, and Eₘ is the redox potential of the ECL moiety. Normally, the product of temperature and change in entropy is approximately 0.1 eV.

The following calculation explains the use of the formula:
Eₐ≦−hc/λ+K+Eₘ (1)
for determining the minimum reducing power of the oxidized, deprotonated amine or amine moiety, and thus in the selection of suitable amines or amine moieties.

For Ru(bpy)₃²⁺ as ECL moiety, the wavelength of emission, λ, is 620 nM. See Tokel N. E., et al., J. Am. Chem. Soc. 94, 2862 (1972). Eₘ is 1.3 V as compared to NHE (NHE is a normal hydrogen reference electrode) and:

$$\begin{matrix}\underline{hc} = \frac{( 4.13 \times {10}^{- 15} eV \text{-} sec ) ( 3 \times {10}^{10} cm \text{/} sec )}{λ6 .2 \times {10}^{- 5} cm} \\ = 2.0 eV ( electron volts ) .\end{matrix} \qquad ( 2 )$$

See Wilkins, D. H., et al., Anal. Chem. Acta. 9, 538 (1953). K is taken to be 0.1 eV. See Faulkner, L. R., et al.,. Am. Chem. Soc. 94, 691 (1972). Substituting these values into equation 1 gives
Eₐ≦−2.0+0.1+1.3 (3)
Eₐ≦−0.6 (4)
Equation 4 implies that the reducing strength of the amine-derived reductant must be equal to or more negative than −0.6 V as compared to NHE. (Note that when referring to potential differences, i.e., Eₐ or Eₘ, the unit of potential is Volts, and the terms hc/λ and K have an energy unit which is eV; however, the conversion from potential difference to eV is unity.)

A wide range of amines and amine moieties are useful in practicing the present invention. Generally, the amine or amine moiety is chosen to suit the pH of the system which is to be ECL analyzed. Another relevant factor is that the amine or amine moiety should be compatible with the environment in which it must function during analysis, i.e., compatible with an aqueous or non-aqueous environment, as the case may be. Yet another consideration is that the amine or amine moiety selected should form a reductant under prevailing conditions which is strong enough to reduce the ECL label.

Amines which are advantageously utilized in the present invention are aliphatic amines, such as primary, secondary and tertiary alkyl amines, the alkyl groups of each having from one to three carbon atoms, as well as substituted aliphatic amines. Triproply amine is an especially preferred amine as it leads to, comparatively speaking, a particularly high-intensity emission of electromagnetic radiation, which enhances the sensitivity and accuracy of detection and quantitation with embodiments in which it is used. Also suitable are diamines, such as hydrazine, and polyamines, such as poly(ethyleneimine). The amine substance in the present invention can also be an aromatic amine, such as aniline. Additionally, heterocyclic amines such as pyridine, pyrrole, 3-pyrroline, pyrrolidine and 1,4-dihydropyridine are suitable for certain embodiments.

The foregoing amines can be substituted, for example, by one or more of the following substituents: —OH, alkyl, chloro, fluoro, bromo and iodo, 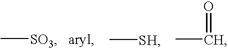
—COOH, ester groups, ether groups, alkenyl, alkynyl, 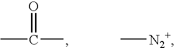
cyano, epoxide groups and heterocyclic groups. Also, protonated salts, for instance, of the formula R₃N—H⁺, wherein R is H or a substituent listed above are suitable. Said substituents are advantageously chosen so as to allow the covalent attachment of said amines to EL species either directly or through linking groups.

Amine moieties corresponding to the above-mentioned amines (substituted or unsubstituted) are also preferred. Tripropyl amine (or an amine moiety derived therefrom) is especially preferred because it yields a very high light intensity. This amine, and the other amines and amine moieties useful in the present invention, work suitably well at pH of from 6 to 9. However, tripropyl amine gives best results at a pH of from 7-7.5. Examples of additional amines suitable for practicing the invention are triethanol amine, triethyl amine, 1,4-diazabicyclo-(2.2.2)-octane, 1-piperidine ethanol, 1,4-piperazine-bis-(ethan-sulfonic acid), and tri-isopropyl amine.

Those of ordinary skill in the art, equipped with the teachings herein, can determine empirically the identity and/or amount of amine or amine moiety advantageously used for the particular system being analyzed, without undue experimentation.

Other suitable reductant/reductant precursor species are oxalate or other organic acid radicals such as pyruvate, lactate, malonate, tartrate and citrate. Alternatively, examples of suitable oxidant/oxidant precursor species are oxidants produced from peroxydisulfate.

It should be noted that “electrochemiluminescence coreactant” and “ECR” as used herein do not include species known as “chemically transformable first compound” (“CTFC”) as that term is used in U.S. Pat. No. 5,643,713 issued Jul. 1, 1997 (which is incorporated by reference). Nevertheless, CR species other than the ECR embodiments discussed above can be also utilized in accordance with the invention.

Thus, the CTFC described in U.S. Pat. No. 5,643,713 is a species which undergoes a structural transformation in response to chemical stimulus, e.g., hydrolysis, as opposed (for instance) to electrochemical stimulus through exposure to electrochemical energy, which transformation alters the measurable luminescence of a detectable ECL compound containing the CTFC in comparison to the measurable luminescence before any such transformation has occurred. For instance, the CTFC can be an enzyme substrate and the chemical stimulus for transformation can be provided by the action of the corresponding enzyme on such substrate. The enzyme substrate can be a beta-lactam, such as (D), and the enzyme its corresponding beta-lactamase. The difference between luminescence of the detectable compound before and after such transformation can be manifest in one of several different ways, as detailed in the chart below:

| measurable luminescence before | measurable luminescence after |
|---|---|
| none | yes (an increase from zero) |
| yes | none (a decrease to zero) |
| yes | yes (an increase from nonzero) |
| yes | yes (a decrease from |
| nonzero). | |

Of course, there must be some luminescence either before or after, or both before and after, any such transformation in order for a perceptible difference to be sensed; if there is no change in luminescence then the species transformed is not a CTFC.

While not wishing to be bound by any particular scientific explanation for the invention's behavior, it is postulated that the ability of the EL-linked CR to act as a reductant or oxidant by intramolecularly donating an electron to or accepting an election from the EL is greater in comparison to the corresponding ability of that same species in the nonconjugated state. Correspondingly, the measured luminescence for the detectable compounds of the present invention is greater in comparison with the measured luminescence of ECL compounds where the CR is not linked to the EL.

Applicants theorize the following explanation as to why, for example, TPA as a nonconjugated reductant generates less electrochemiluminescence than TPA as a conjugated reductant. The nonconjugated TPA must first diffuse through solution to become sufficiently proximate to the EL and then intermolecularly donate an electron thereto. Moreover, during this diffusion process, the nonconjugated TPA may react with available species other than the EL because the CR is a reactive, radical species. In direct contrast, the conjugated TPA does not have to diffuse through the solution as a free species; the conjugated TPA need only intramolecularly donate an electron to the EL linked to it.

Linkage.

The linkage between the CR and the EL comprises a linker group or a chemical bond that links one or more CRs to one or more ELs. In one example, one end of the linkage has a bond between a linker group and the EL (for example, to a ligand of an EL containing a coordinated metal) while another end of the linkage has a bond between a linker group and the CR. In another example, the linkage has bonds to one or more ELs and one or more CRs. The linkage may also comprise one or more linking groups for attachment of biomolecules such as proteins, nucleic acids, cells and the like. Examples of appropriate linking groups include NHS-esters, carboxylic acids, amines, thiols, disulfides, maleimide, hydroxy and the like; these and other functional groups that can be used to attach biomolecules are, in and of themselves, well known in the art.

In some embodiments, the linkage may comprise a linking group such as a polymer, a polypeptide chain, a polynucleic acid strand, a polysaccharide, an oligo-ethylene glycol group, a fiber or the like. These linking groups are, in and of themselves, known in the art and commonly used as linking moieties or spacers.

The linking group may also comprise a ligand on an EL containing a coordinated metal. For example, the linking group may comprise a functionalized bipyridyl group such that the bipyridyl group is linked via appropriate functional groups and/or spacers to a CR.

Certain attributes of the linker group are advantageous so that its presence does not undesirably interfere with the operability of the invention. Specifically, the linking group during the contemplated practice of the invention preferably: (i) does not prohibit the electrochemical reactions required for ECL; (ii) does not prohibit the overall electrochemiluminescence mechanism.

Other attributes, e.g., the length of the linking group and the nature of the bonds within such length, preferably (i) allow and permit the appropriate electron transfer reactions to occur; and (ii) do not prevent any necessary reaction from occurring. Electron transfer between the ECR and the EL (or between any two or more species) can occur intermolecularly or intramolecularly. Advantageously, the linkage allows for efficient intramolecular electron transfer between the ECR and the EL.

“Intramolecular” transfers include transfers between a donating compound (e.g., the CR) and a corresponding receiving compound (e.g., the EL) which are linked to each other, e.g. through a linking group. The term “intramolecular transfer” encompasses both transfer through bonds and through space. The linking group must allow and permit at least one these two types of intramolecular transfer.

For intramolecular transfer through bonds, the linker group desirably provides sufficient delocalized, conductive electrons (e.g., conjugated π-systems) to enable the electrons to travel efficiently through the bonds of the linking group. For intramolecular electron transfer through space, the linkage desirably enables the reactants (e.g., the ECR and the EL or 2 ECRs) to approach in close proximity (such that electron transfer is efficient.)

For example, the linker group desirably enables the CR to approach in relatively close proximity the central metal cation of the EL. The linker group is advantageously long enough and stereochemically flexible enough so that the CR attached to the far end of the linker group can swing back towards the metal cation and then the electron can intramolecularly transfer through the space then separating the CR and the EL. An additional consideration bearing on the appropriate length of the linker group is that it advantageously need not be so long that the frequency of the described swinging around effect (which effect is thought to be necessary for intramolecular transfer through space) significantly decreases. In the case of an excessively long linker group, the amount of luminescence produced could be decreased.

Linkages in accordance with the invention provide several advantages. First, the linkage maintains the CR in close proximity to the EL: electron transfer between the CR and the EL can thus be very efficient. In particular, it can eliminate inefficiencies that result from diffusion of free species (e.g., ECR) through solutions (an inevitable process when the ECR and EL are not kept in proximity by a linkage group). The increased efficiency of electron transfer afforded by a linkage group allows for more rapid, efficient generation of the excited, luminescent forms of the EL and therefore higher ECL signals in the practice of the present invention when compared to systems that do not use linkages between CRs and ELs.

In some embodiments, the linkage of the EL and the CR advantageously promotes the catalytic oxidation or reduction of the CR by the EL. An example is the following ECL mechanism: 1) oxidation of EL to EL+ at an electrode; 2) electron transfer from the EL+ to the CR to form a strong reductant CR+ and to regenerate EL; 3) reoxidation of EL to EL+ at an electrode and 4) the reaction of the strong reductant CR+ and the oxidated label EL+ to promote EL to an electronically excited state that can luminesce.

Once in possession of the teachings herein those of ordinary skill in the art can select suitable linker groups. For instance, Vol. 136, Methods in Enzymology, K. Mosbach, Ed., pp. 3-30, Academic Press, NY (1987) discloses a series of “spacer molecules” for immobilized active coenzymes, including NAD and ATP. The spacer molecules of this article, which article is fully incorporated by reference, are examples of such suitable candidates.

The above analysis, in connection with the disclosure herein, teaches attributes of the covalent linkage sufficiently detailed to enable the skilled worker to practice the present invention. Thus, the skilled worker can select appropriate candidates as linking groups and determine, by routine experimentation, those which do and do not work.

(C) The Electrochemilumine Scent Species.

The third portion of the detectable compounds is the EL. These have been reported in the literature. See, for example, the references and issued U.S. patents previously incorporated by reference. The attributes and identities of such ELs, in and of themselves, are known to skilled workers.

Accordingly, once in possession of the teachings herein, the skilled worker can practice the present invention by adapting the existing knowledge of EL species. Notwithstanding this, applicants provide guidelines for selecting EL species operative in the present invention.

As previously indicated, minor variations in the oxidation state the EL species are permitted. Thus, changes in formal redox state of the EL species due to, for example, electrochemical oxidation or reduction, and intramolecular reduction or oxidation, as well as differences between excited/nonexcited states, are encompassed by the term “EL” and such changes represent acceptably varying forms of the EL.

EL can also refer to a label that is destroyed during the ECL process. For example, the generation of ECL from luminol is believed to involve oxidation at an electrode and reaction with a cofactor to give an intermediate species that then decomposes to a high energy luminescent species.

In a preferred embodiment the EL contains a coordinated metal (herein sometimes “ELM”). Some examples include transition metal polypyridyl complexes and lanthanide chelates. The following formula depicts suitable coordinate complexes for use in the present invention:
M(L¹)ₐ(L²)_{b}(L³)_{c}(L⁴)_{d}(L⁵)ₑ(L⁶)_{f}
wherein
M is a central metal cation comprising ruthenium or osmium;
L¹ through L⁶ are each ligands of M, each of which may be monodentate or polydentate, and each of which may be the same or different from each other;
a through e are each 0 or 1;
   provided that the ligands of M are of such number and composition that the compound can be induced to electrochemiluminesce; and further provided that the total number of bonds provided by the ligands to the central metal cation M equals the coordination number of M.

In the practice of the present invention, especially preferred ELM species are those which include coordinate complexes wherein the central metal cation is ruthenium (Ru) or osmium (Os). Particularly preferred ELM species are those comprising Ru(bpy)₃⁺². Other ELM species are:
ruthenium complexes such as Ru(bpy)₃²⁺ (bpy-2,2′-bipyridine), Ru(bpy)₃²⁺-oxalate, Ru(bpy)₃²⁺-persulfate, Ru(bpy)₃²⁺-tripropylamine, Ru(bpz)₃²⁺(bpz-bipyrazine), Ru(o-phen)₃²⁺ species (o-phen 1.10-phenanthroline), Ru(4-vinyl-4′-methyl-2.2′-bipypyridine)₃²⁺ polymer, Ru(4.4′-diphenyl-2.2′-bipyridine)₃²⁺ and Ru(4.7-diphenyl-1.10-phenanthroline)₃²⁺;
osmium complexes such as Os(bpy)₃²⁺, Os(o-phen)₃²⁺; Os[(4.4′-distyryl-2.2′-bipyridine)₂ bis-1.2-diphenylphoninoethane]²⁺, Os(bpz) 3²⁺;
platinum and palladium complexes such as Pd(O) and Pt(O) complexes of dibenylidinaceone and tribenzylidineacetylacetone, Pt[2-(2-thienyl)-2-pyridine]₂, Pt₃(diphosphonate)₄⁴⁻, Pd and Pt tetraphenylporphyrins, Pt(quinolin-8-olate)₂;
molybdenum and tungsten clusters such as Mo₂Cl₄(PMe₃)₄, Mo₆Cl₁₄²⁻, Mo₆Cl₁₄²⁻ and W₆X₈Y₆²⁻, where (X.Y—Cl.Br.l;X—Cl.Y—Br;X-1.Y—Br); and
other inorganic compounds such as Tb(TTFA)₃(o-phen), Tb(TTFA)₄⁻, Eu(TTFA)₃(o-phen) (TTFA-thenolytrifluroacetonate), Eu¹¹¹ dibenzoylmethide and dinaphthoylmethide, silicon phthalocyanine and naphthalocyanine, Cr(bpy)₃²⁺, Re(CO)₃Cl(o-phen), binuclear Ir¹ complexes, Ir(2-phenylpyridine-C²,N¹)₃ complex, [Cu(pyridine)I]₄, uranylsulfate in concentrated sulfuric acid.

Other types of EL species can be used in the practice of the present invention, that is, practice of this invention does not need to be limited to metal cation-liquid complexes. Organic compounds which are electrochemiluminescent can constitute suitable EL species in some embodiments. For example, the EL species may comprise a substituted or unsubstituted polyaromatic molecules. Typically, organic EL species include polyaromatic hydrocarbons, such as 9,10-diphenylanthracene, rubrene, phenanthrene, pyrene, poly(vinyl-9,10-diphenylanthracene)polymer, trans-stibelene derivatives, donor-substituted polyaromatic hydrocarbons;
mixed systems such as 9,10-diphenylanthracene (9,10-DPA-)-1,4-dihydropyridines, 9,10-DPA-N,N,N′,N′-tetramethyl-p-phenylenediamine (TMPD), 9,10-DPA-halogen ions, 9,10-DPA-9,10-dichloro-9,10-dihydro-9,10-DPA, rubrene-TMPD, rubrene-amines, water or dimethylformamide, tetracene-TMPD, aromatic hydrocarbons-persulfate, aromatic hydrocarbons-tetraphenylporphins, aromatic hydrocarbons-tetrathiafulvalene, fluoranthrene-10-methylphenothiazine, thianthrene-2,5-diphenyl-3,4-oxadiazole, aryl derivatives of N,N-dimethylanillne, aryl derivatives of isobenzofurans and indoles.

In other embodiments, the EL species can comprise a fluorescent dye (e.g., fluorescein). The EL species can also be a chemiluminescent label such as lumisol, isoluminol and/or other Derivatives.

(d) Illustrative Uses of the Detectable Compounds.

One of ordinary skill will readily understand that the broader disclosures of the application encompasses the use of electrochemiluminescence in a number of assay formats. Such assays include immunological binding assays, DNA/RNA hybridization assays, Southern hybridization assays, dot blotting assays, Western blot assays, or any other type of assay in which a chemical moiety is bound, or is capable of being bound to an analyte. Some of these assays are described in further detail below:

DNA/RNA hybridization exploits the ability of complementary sequences in single-stranded DNAs or RNAs to pair with each other to form a double helix (i.e., the binding event, wherein the DNA/RNA sequences are said to be binding partners of one another). The two polynucleotide strands are held together in an antiparallel configuration by hydrogen bonding between the bases G and C and between A and T (or U). Hybridization may be effected in solution, or on a filter, wherein one of the nucleic acid components of the hybridization is immobilized on a membrane filter.

Southern hybridization typically involves the separation of restriction fragments of DNA on agarose, transference and fixation of the fragments to a filter (blotting), and hybridization with a labelled DNA or RNA probe containing the required sequence. Similarly, Northern hybridization is used to analyze RNA sequences, and it involves the electrophoretic separation of RNA on agarose, transference and fixation of the RNA to a membrane filter, and hybridization with labeled RNA or DNA probes.

Dot blotting typically involves the detection of RNA or DNA sequences, wherein the samples are dotted directly onto the membrane filters without prior electrophoretic separation, and hybridization is carried out as in Northern or Southern blotting.

In situ hybridization is used to detect and locate specific DNA or RNA sequences in tissues or on chromosomes. A labeled DNA or RNA probe of the required sequence typically is applied to fixed tissue or chromosomal preparations, where it hybridizes with any complementary sequences present.

Western blotting is a method for detecting one (or more) specific proteins in a complex protein mixture by monitoring the affinity of the components of the mixture for the corresponding antibody of the protein of interest. The procedure requires the fractionation of a protein mixture by electrophoresis, transference and immobilization of the mixture onto a solid support, and incubation of the membrane with a solution containing an antibody raised against the protein of interest. In this instance, the protein is the analyte of interest, the antibody raised against that protein is the binding partner, and the binding event takes place by contacting the membrane with the antibody. Detection of the analyte of interest involves detection of the presence of the antibody complexed to the protein on the solid support.

Enzyme assays, wherein the analyte of interest is an enzyme, can involve contacting the solution containing the enzyme with a labeled substrate for the enzyme, i.e., the binding partner of the enzyme, and monitoring a binding event by detecting the presence of the label in solution. In addition, the binding event may be followed by turnover by the enzyme of the labeled substrate to product, in which case, the detection step may involve monitoring the presence of labeled product.

Another use of the detectable compounds of the invention is in kits specifically designed to implement assay methods incorporating the invention. The assaying kits comprise a plurality of sample solutions each containing a known amount of a particular detectable compound differing from the amount of such compound in any other of the solutions. Using this plurality of solutions, one of ordinary skill can develop a calibration standard.

### (E) EXAMPLES

Notwithstanding the previous detailed description of the present invention, applicants below provide specific examples solely for purposes of illustration and as an aid to understanding the invention. Particularly with respect to the protection to which the present invention is entitled to, these examples are both nonlimiting and nonexclusive. Accordingly, the scope of applicants' invention as set forth in the appended claims is to be determined in light of the teachings of the entire specification without incorporating in such claims the specific limitations of any particular example.

### Example 1

### Preparation of Covalent Conjugates of Ru(bpy)₃²⁺ and ECL Co-Reactants

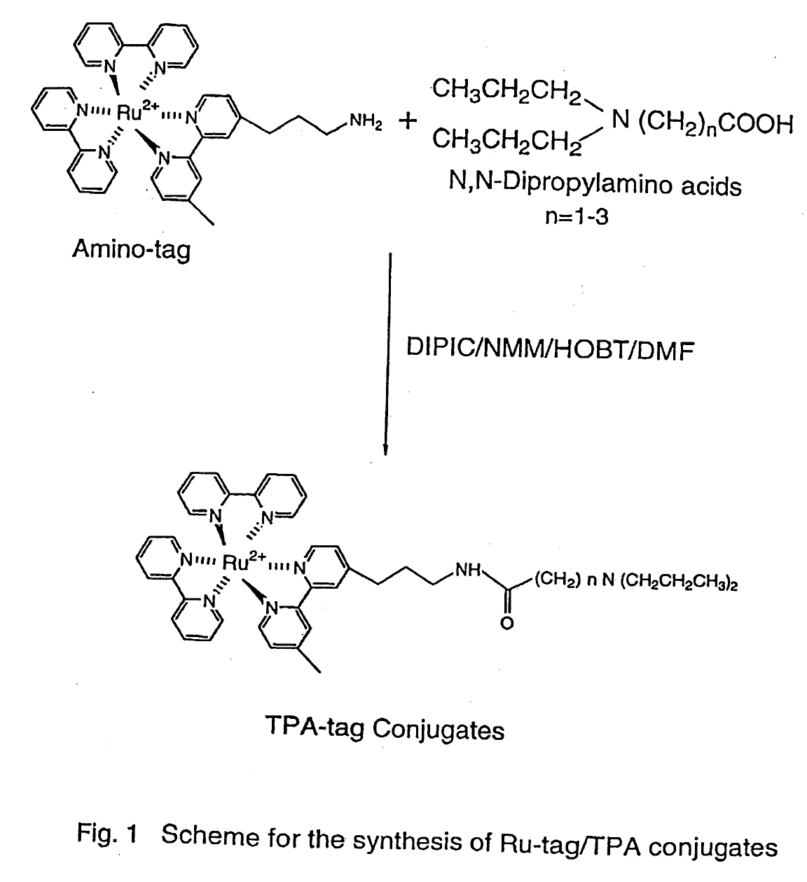

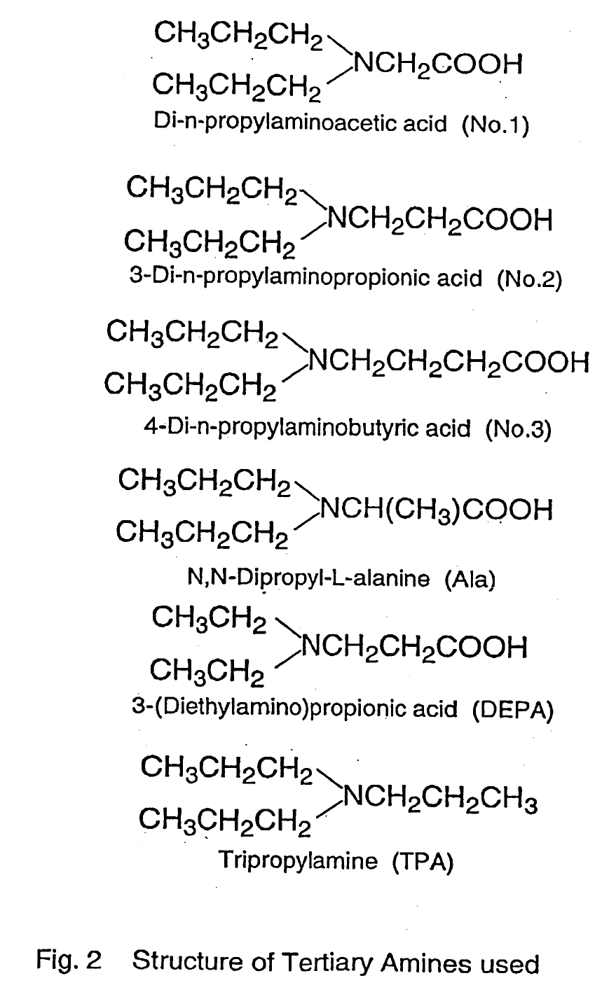

A series of four conjugates were prepared each from a derivative of Ru(bpy)₃²⁺ and an amine. The reaction between the primary amine derivative of Ru(bpy)₃²⁺ and a series of three N,N-dipropylamino acids to give conjugates (1-3) is shown in FIG. 1. In addition, the conjugate Ru(bpy)₃²⁺-DPA) between Ru₃²⁺ and N,N-dipropyl-L-alanine, was formed and tested. The structures of the amines that were conjugated are shown in FIG. 2, as are the structures of two other amines, 3-(diethylamino)propionic acid and TPA, which were investigated in non-conjugated form. The preparation of the conjugates is described below.

Preparation of the N,N-dipropylaminocarboxylic acids by hydrolysis of the corresponding nitrites. In all cases except for N,N-dipropyl-L-alanine, preparation of the N,N-dipropylamino acids required the preparation of the carboxylic acid derivative (—COOH, not commercially available) from the nitrile derivative (—CN, commercially available, Lancaster Synthesis). This conversion was performed by hydrolysis of each compound (3 mL) in a mixture of deionized water (10 mL) and concentrated sulfuric acid (10 mL). The reaction mixture was refluxed for 7 hours. The mixture was then diluted with water and BaCO₃ was added to precipitate the sulfate. Barium sulfate was removed by filtration. Activated charcoal was added to the liquid phase and the solution pH was adjusted to 1 with HCl, heated, and the charcoal was removed by filtration. The liquid phase was extracted four times with 60 mL CH₂Cl₂ and dried using a rotary evaporator. The resulting sticky liquid was dissolved in CH₃CN/CH₂Cl₂₁ (1:4) and loaded on a silica column. The compounds were eluted with 100 mL CH₃CN/CH₂Cl₂, (2:8), 100 mL CH₃CN/CH₂Cl₂, (3:7), 50 mL CH₃CN/CH₂Cl₂, (4:6), 50 mL CH₃CN/CH₂Cl₂₁ (7:3), 50 mL CH₃CN/CH₂Cl₂, (8:2), and 350 mL CH₃CN/CH₂Cl₂, (1:9). Each fraction was tested by TLC and the fractions containing the major band were pooled and dried by rotary evaporation. Finally, the solid was dissolved in 6 mL concentrated HCl and the HCl was evaporated to yield the hydrochloride of the product. Yields typically were 0.3 to 0.8 g.

Preparation of the conjugated between Ru(bpy)₃²⁺-amine and N,N-dipropylaminoacetic acid. N,N-dipropylaminoacetic acid (38 g) and 1-hydroxybenzotriazole (HOBT, 25 mg) were dissolved in approximately 400 μL anhydrous dimethylformamide (DMF). N,N-Diisopropylcarbodiimide (DIPCDI, approximately 35 μL) was added, and the solution, which turned milky white within 5 minutes, was stirred for one hour at room temperature. The primary amine derivative of Ru(bpy)₃²⁺ (FIG. 1) (22 mg) was then added to the reaction flask with the aid of another 50 μL of DMF which was used as a rinse. N-Methylmorpholine (NMM, approximately 30 μL) was added and the reaction was allowed to proceed overnight at room temperature.

The reaction mixture was purified by ion exchange, size exclusion, and a second ion exchange chromatography procedures. First, the reaction mixture was loaded on a column of SP Sephadex C25 cation exchange media (Sigma). The column was eluted with deionized water, followed by 50 mM Trifluoroacetic acid (TFA), and 500 mM TFA. The major visible band was concentrated to dryness using a rotary evaporator, dissolved in deionized water and eluted on a Biogel P-2 size exclusion column (BioRad). The eluted product was finally re-purified on a SP Sephadex C25 column using 50, 100, 200 and 300 mM TFA as eluting solvents. The product was dried using a rotary evaporator.

Preparation of the conjugate between Ru(bpy)₃²⁺-amine and N,N-diropyl-4-aminobutyric acid. The conjugate was prepared as described above, except that 40 mg of N,N-dipropyl-3-aminopropionic acid was used instead of N,N-dipropylaminoacetic acid.

Preparation of the conjugate between Ru(bpy)₃²⁺-amine and N,N-diropyl-4-aminobutyric acid. The conjugate was prepared as described above, except that 41 mg of N,N-dipropyl-4-aminobutyric acid was used instead of N,N-dipropylaminoacetic acid.

### Example 2

### ECL Properties of Various Tertiary Amines Prior to Conjugation with Ru(bpy)₃²⁺

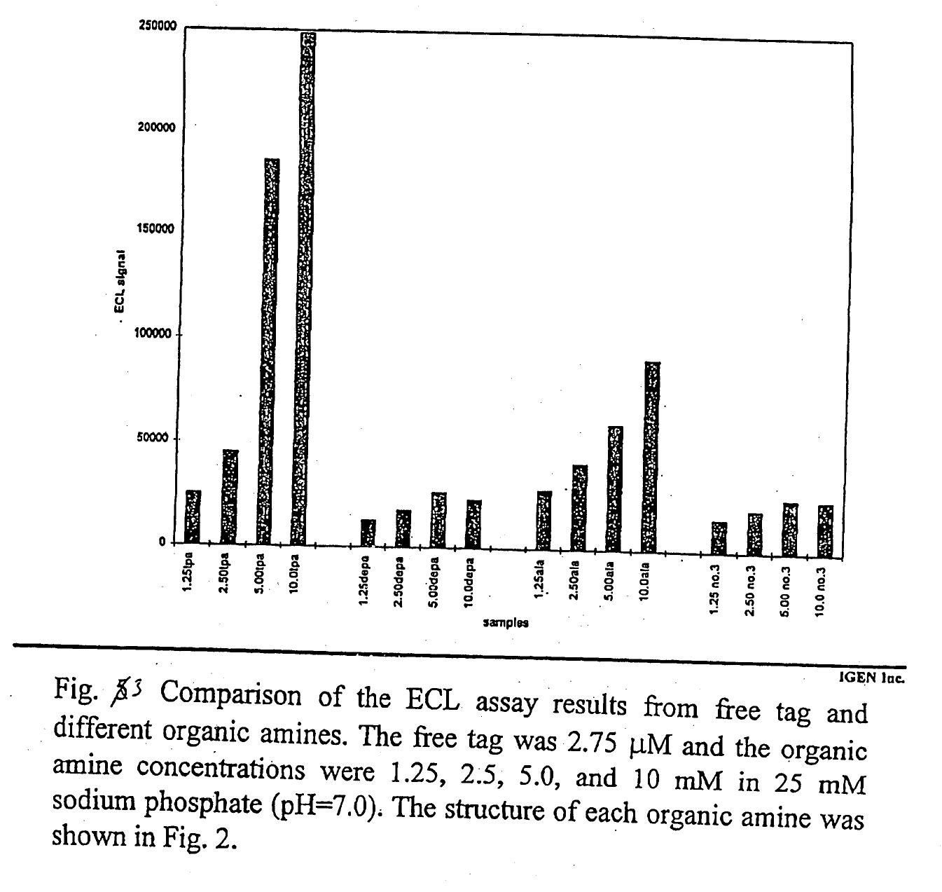

The ECL of (non-conjugated) mixtures of 2.75 μM RU(bpy)₃²⁺ and various concentrations (1.25, 2.50, 5.00 and 10.00 Mm) of four tertiary amines was measured (FIG. 3). In comparison to TPA, all gave weaker ECL light emission. After TPA, N,N-dipropyl-L-alanine (“ala” in FIG. 3) gave the most light, followed by both N,N-diethyl-3-aminopropionic acid (“depa” in FIG. 3) and N,N-dipropyl-4-aminobutyric acid (“no. 3” in FIG. 3), which gave similar emissions. The ECL measurements were made in an ORIGEN Analyzer (IGEN) in 25 Mm sodium phosphate, Ph 7.0.

### Example 3

### Relative ECL Efficiency of Four RU(bpy)₃₂₊-Co-Reactant Conjugates

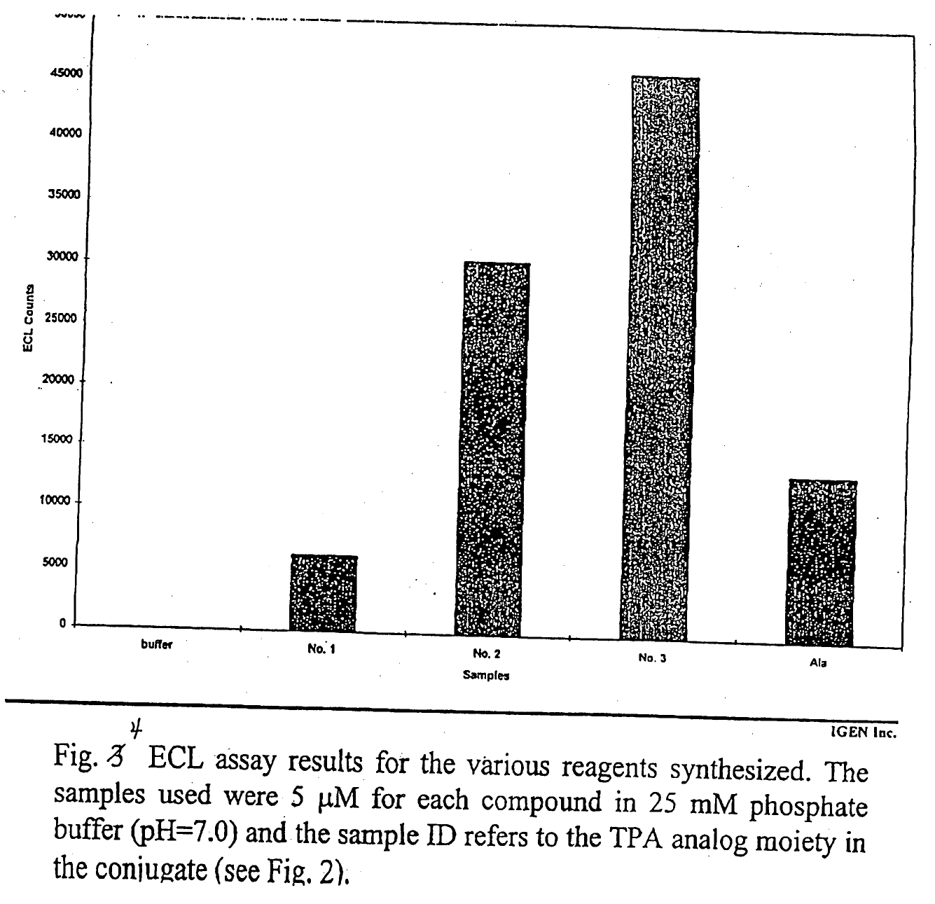

FIG. 4 shows the ECL (ORIGEN Analyzer, IGEN) of 5.0 μM solutions of four RU(bpy)₃₂₊-co-reagent conjugates. These are described as No. 1 (Ru(bpy)₃²⁺ conjugate of N,N,-dipropylaminoacetic acid), No. 2 (Ru(bpy)₃²⁺-N,N,-dipropyl-3-aminopriopionic acid), No. 3 (Ru(bpy)₃²⁺-N,N,-dipropyl-4-aminobutyric acid) and Ala (Ru(bpy)₃²⁺-N,N,-dipropyl-L-alanine). The assays were carried out in 25 Mm sodium phosphate, Ph 7.0. These results show that, in terms of ECL light emission efficiency, No. 3.>No. 2>No. 1, indicating that, in these compounds, the longer linker allowed for more efficient ECL emission. Although Ala is not a structural homolog to compounds No. 1-No. 3, its ECL efficiency fits the pattern in that the ECL efficiency as well as the distance between Ru(bpy)₃²⁺ and the tertiary amine is roughly the same as in the No. 1 conjugate.

### Example 4

### Quantitation of the Increase in ECL Efficiency Obtained by Conjugating Ru(bpy)₃²⁺ to a Tertiary Amine Co-Reactant

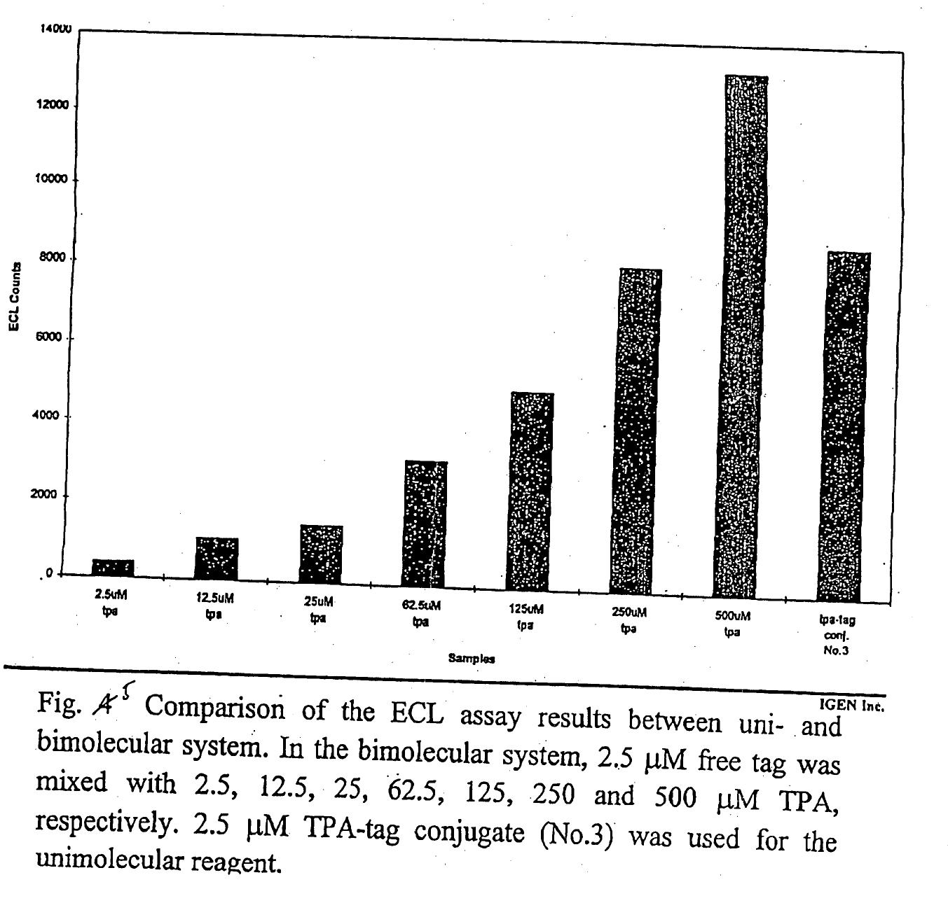

FIG. 5 shows a comparison of the ECL (ORIGEN Analyzer, IGEN) of a 2.5 μM solutions of the Ru(bpy)₃²⁺ conjugate of N,N,-dipropyl-4-aminobutyric acid (No. 3) with mixtures of 2.5 μM free Ru(bpy)₃²⁺ and various concentrations of TPA. These data show that the ECL seen with the 2.5 μM of the conjugate is approximately the same as that seen from a mixture of 2.5 μM free Ru(bpy)₃²⁺ and 250 μM TPA (100 times more light per tertiary amine molecule). Moreover, because the efficiency of TPA as an ECL co-reagent is approximately 10 times greater than that of N,N,-dipropyl-4-aminobutyric acid (see FIG. 3), it appears that there is approximately a 1000-fold increase in ECL efficiency of N,N,-dipropyl-4-aminobutyric acid upon conjugation to Ru(bpy)₃²⁺.

The scope of the patent protection which the present invention is entitled to is not limited by the preceding text. Rather, the present invention is defined by the claims appended hereto and all embodiments falling thereunder.

1. (canceled)

2. A method of generating an electrochemiluminescent emission, which comprises exposing compound comprising an electrochemiluminescent label linked to an electrochemiluminescence coreactant, to conditions suitable for inducing electrochemiluminescence, wherein said electrochemiluminescent label comprises a coordinate complex of a metal.

3-8. (canceled)

9. The method of claim 2 wherein,
(a) said coreactant can be oxidized to form a reductant or reduced to form an oxidant; and
(b) on exposure of said compound to electrochemical energy sufficient to form said reductant or said oxidant, said reductant or oxidant reacts with said label so as to cause said label to emit electrochemiluminescence.

10. The method of claim 9, wherein the electrochemiluminescent label linked to the coreactant has the formula:

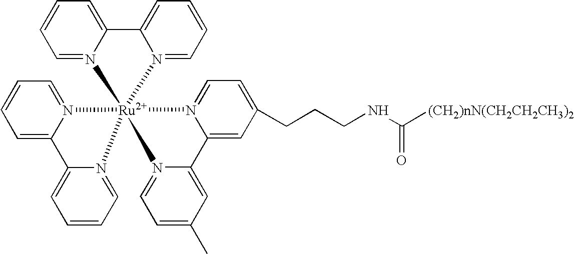

11. The method of claim 9, wherein said electrochemiluminescent label is linked to said coreactant by a linkage which comprises one or more linking groups for attaching biomolecules.

12. The method of claim 9, wherein said coreactant is an amine.

13. The method of claim 9, wherein said coreactant comprises an aliphatic tertiary amine moiety.

14. The method of claim 9, wherein said coreactant comprises a dipropyl amine moiety.

15. The method of claim 9, wherein said coreactant is an N,N-dipropyl amino acid.

16. The method of claim 9, wherein said coreactant is NADH.

17. The method of claim 9, wherein said coreactant is the hydrolyzed form of a β-lactam antibiotic having a hydrolyzed β-lactam bond.

18. The method of claim 9, wherein said electrochemiluminescent label comprises ruthenium, osmium, or rhenium.

19. The method of claim 9, wherein said electrochemiluminescent label and said coreactant are linked by an amide bond.

20. The method of claim 9, wherein said ECL label and said coreactant are linked via a functional group of said ECL label or said coreactant.

21. The method of claim 9, wherein said ECL label and said coreactant are linked via a linker comprising a polymer, a polypeptide chain, a polynucleic acid, a polysaccharide, an oligo-ethylene glycol group, or combinations thereof.

22. The method of claim 9, wherein said ECL label and said coreactant are linked via a linkage comprising one or more linking groups selected from the group consisting of NHS-esters, carboxylic acids, amines, thiols, disulfides, maleimides, hydroxides, or combinations thereof.

23. The method of claim 9, wherein said ECL label is oxidized by exposure to electrochemical energy and said coreactant is a reductant or a reductant precursor.

24. The method of claim 9, wherein said ECL label is reduced by exposure to electrochemical energy and said coreactant is an oxidant or oxidant precursor.

25. A method of generating an electrochemiluninescent emission, which comprises exposing an electrochemiluminescent label selected from 9,10-diphenylanthracene, rubrene, phenanthrene, pyrene, poly(vinyl-9,10-diphenylanthracene)polymer, and trans-stibelene derivatives, said label being linked to an electrochemiluminescence coreactant, to conditions suitable for inducing electrochemiluminescence.

26. The method of claim 26 wherein,
(a) said coreactant can be oxidized to form a reductant or reduced to form an oxidant; and
(b) on exposure of said compound to electrochemical energy sufficient to form said reductant or said oxidant, said reductant or oxidant reacts with said label so as to cause said label to emit electrochemiluminescence.

27. The method of claim 27, wherein said electrochemiluminescent label is linked to said coreactant by a linkage which comprises one or more linking groups for attaching biomolecules.

28. The method of claim 27, wherein said coreactant is an amine.

29. The method of claim 27, wherein said coreactant comprises an aliphatic tertiary amine moiety.

30. The method of claim 27, wherein said coreactant comprises a dipropyl amine moiety.

31. The method of claim 27, wherein said coreactant is an N,N-dipropyl amino acid.

32. The method of claim 27, wherein said coreactant is NADH.

33. The method of claim 27, wherein said coreactant is the hydrolyzed form of a β-lactam antibiotic having a hydrolyzed β-lactam bond.

34. The method of claim 27, wherein said electrochemiluminescent label and said coreactant are linked by an amide bond.

35. The method of claim 27, wherein said ECL label and said coreactant are linked via a functional group of said ECL label or said coreactant.

36. The method of claim 27, wherein said ECL label and said coreactant are linked via a linker comprising a polymer, a polypeptide chain, a polynucleic acid, a polysaccharide, an oligo-ethylene glycol group, or combinations thereof.

37. The method of claim 27, wherein said ECL label and said coreactant are linked via a linkage comprising one or more linking groups selected from the group consisting of NHS-esters, carboxylic acids, amines, thiols, disulfides, maleimides, hydroxides, or combinations thereof.

38. The method of claim 27, wherein said ECL label is oxidized by exposure to electrochemical energy and said coreactant is a reductant or a reductant precursor.

39. The method of claim 27, wherein said ECL label is reduced by exposure to electrochemical energy and said coreactant is an oxidant or oxidant precursor.
